(19) United States
(12) Patent Application Publication
(10) Pub. No.: US 2026/0335098 A1
(43) Pub. Date: Oct. 29, 2026

(54) BONE JOINT IMPLANT ESPECIALLY SUITABLE FOR A CARPOMETACARPAL (CMC) JOINT

(71) Applicant: LOCI ORTHOPAEDICS LIMITED, Galway (IE)

(72) Inventors: Brendan BOLAND, Galway (IE); Fiona MANGAN, Galway (IE); Gerry CLARKE, Galway (IE)

(73) Assignee: LOCI ORTHOPAEDICS LIMITED, Galway (IE)

(21) Appl. No.: 19/103,963

(22) PCT Filed: Jun. 13, 2023
(86) PCT No.: PCT/EP2023/065845

§ 371 Date: Feb. 14, 2025

(30) Foreign Application Priority Data
Aug. 23, 2022 (EP) 22191807.1
Nov. 8, 2022 (EP) 22206180.6

(51) Int. Cl. A61F 2/42; A61B 17/16; A61B 17/32; A61B 17/56; A61F 2/30; A61F 2/46

(52) U.S. Cl. CPC A61F 2/4261; A61B 17/1659; A61B 17/1686; A61B 17/32; A61B 17/56; A61F 2/30771; A61F 2/46; A61B 2017/564; A61F 2002/30112; A61F 2002/30324; A61F 2002/30332; A61F 2002/30433; A61F 2002/30616; A61F 2002/3093; A61F 2002/4271; A61F 2310/00023; A61F 2310/00029; A61F 2310/00161; A61F 2310/00407

(57) ABSTRACT

An implant apparatus has an implant (100, 600) with an intramedullary part (101, 601) and a proximal part (102, 602) having a proximal articular surface (103, 603) which is curved in a generally concave manner for direct contact and sliding engagement with a generally convex distal surface (16) of a proximal bone. The proximal articular surface is curved in an asymmetric manner with a part of the proximal surface extending more proximally on one lateral side of an axis of the stem. The proximal part (102, 152) has rounded comers (104, 154, 604) such that there is no edge or point loading when in contact with a bony surface. The proximal articular surface accommodates sub-luxation of the joint, and in some cases the apparatus has multiple implants or proximal parts of implants so that a particular articular surface can be chosen to suit the degree of sub-luxation.

The present invention relates to bone joint implants in animals or humans. It applies especially, but not exclusively to an implant to function in any subluxed or incorrectly aligned joint such as the human carpometacarpal (CMC) joint.

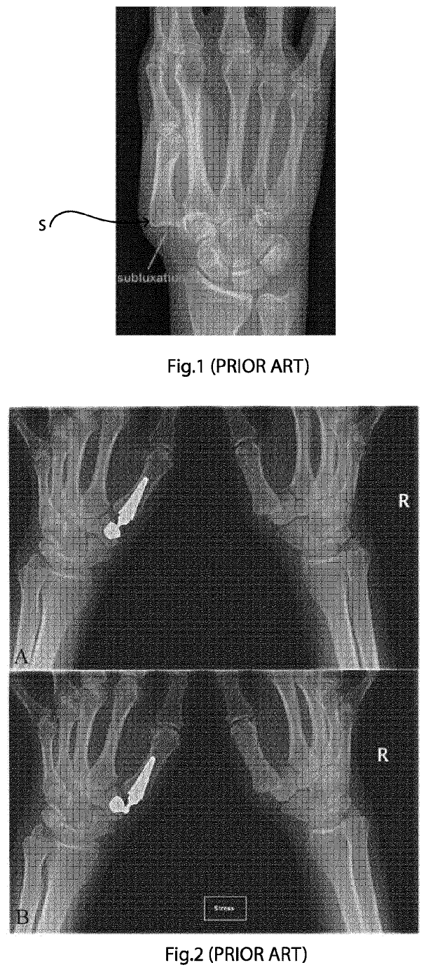

The thumb base or carpometacarpal (CMC) joint is a dual axis saddle joint made up of the first metacarpal bone and the trapezium bone. One axis of motion, the abduction-adduction axis, traverses the proximal base of the first metacarpal, while the other, the extension-flexion axis, traverses through the trapezium bone. There is additionally a coupled internal-external rotation motion of the joint. These are illustrated for example in our prior published PCT specification WO2020/193078, see for example FIG. 1(b) of that document.

These complex biomechanics provide the joint with a wide range of motion which facilitates the function of this mobile joint, but this mobility is also associated with an inherent instability of the joint. This instability is moderated by a complex of ligamentous structures which stabilise the joint.

In the healthy CMC joint, there is alignment of the trapezium and metacarpal bones. The proximal portion of the metacarpal sits directly over the trapezium with the saddle shaped elements of each one mating to enable the joint to function normally.

However, either as the cause of, or a result of, arthritis of the joint, the joint can become subluxed i.e. the metacarpal bone no longer sits on the trapezium bone, see subluxation S in FIG. 1, [2]. It is thought that over time the ligaments that support the joint tend to weaken and fail. This causes the forces transmitted through the joint to put further pressure on the supporting ligaments as the joint is no longer correctly aligned, most markedly on the dorsal aspect of the joint. As forces are transmitted through the joint and the weakened dorsal ligaments, it is thought that this causes these ligaments to weaken further, beginning as failure cycle where, as the ligaments weaken, they are less able to withstand the stresses put though them, causing them to weaken further and hence the joint to sublux in a dorsal direction.

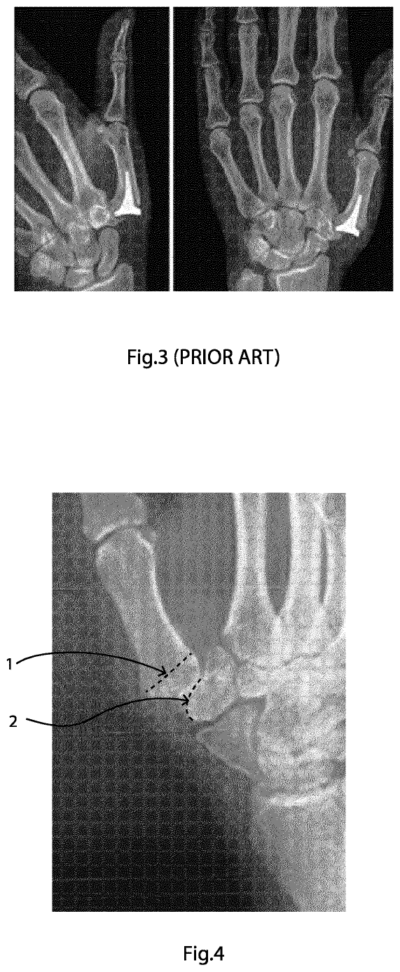

Arthritis of the CMC joint is associated with pain and decreased hand function. Many implants have been developed in an effort to treat the clinical effects of thumb base joint arthritis. These can be broadly defined as total arthroplasty implants (implants which have one portion anchored in the metacarpal and another anchored in the trapezium with a connection between each element) and hemi-arthroplasty implants (implants which sit on one side of the joint only). An example of a subluxed full arthroplasty is shown in FIG. 2, showing in the bottom image that the implant has failed dorsally with the head displacing from the cup [3]. An example of a subluxed hemi arthroplasty is shown in FIG. 3 [4].

After implants have been placed in the joint, the joint is again aligned in a normal axis where the metacarpal sits directly over the trapezium, as noted in the upper image of FIG. 2. This is facilitated by a reconstruction of the joint capsule and surrounding ligaments in an effort to return the joint to the pre-diseased state. However, when the failure modes of such implants are analysed, the most common failure is dorsal subluxation of the implant and dislocation of the implants from its intended position. It is suggested that in many patients who have an underlying deficiency in the strength of these ligaments it is not possible to adequately reconstruct the dorsal aspect of the joint capsule. This results in the joint reverting to the pre-implantation state of subluxation, with a resultant alteration in the biomechanics of the joint and a failure of the implant.

US2014/194999 (Skeletal Dynamics) and US2011/087297 (Skeletal Dynamics) describe implants which have a proximal C-shaped part for engaging and surrounding part of the trapezium.

The present invention is directed towards providing an improved bone joint implant to address these problems.

### REFERENCES

[1] Crisco JJ, Halilaj E, Moore DC, Patel T, Weiss AP, Ladd AL. In Vivo kinematics of the trapeziometacarpal joint during thumb extension-flexion and abduction-adduction. J Hand Surg Am. 2015; 40(2): 289-296. doi: 10.1016/j.jhsa.2014.10.062.
[2] Halilaj E, Moore DC, Patel TK, Ladd AL. Weiss A-PC, Crisco JJ. Early osteoarthritis of the trapeziometacarpal joint is not associated with joint instability during typical isometric loading. J Orthop Res. 2015; 33:1639-1645 10.1002/jor.229364591106.
[3] Goubau, Jean & Goorens, chul ki & Van Hoonacker, Petrus & Berghs, Bart & Kerckhove, D & Scheerlinck, T. (2013). Clinical and radiological outcomes of the Ivory arthroplasty for trapeziometacarpal joint osteoarthritis with a minimum of 5 years of follow-up: A prospective single-centre cohort study. The Journal of hand surgery, European volume. 38. 10.1177/1753193413488494.
[4] Caudwell M., Bayne G., Page R.S. Anatomic pyrocarbon hemiarthroplasty for thumb carpometacarpal osteoarthritis in patients under 65 years: mid term results. J Hand Surg Asian Pac Vol. 2018; 23:469-473 https://doi.org/10.1142/S2424835518500443

### SUMMARY OF THE INVENTION

We describe an implant apparatus comprising an implant comprising:
an intramedullary part configured for intramedullary engagement in a human metacarpal, and
a proximal part,
wherein said parts are fixed together or integral, said proximal part has a proximal articular surface which is curved with a concave portion for direct contact and sliding engagement with a distal surface of a proximal bone such as the trapezium,
wherein said articular surface is curved in an asymmetric manner with a part of the articular surface extending more proximally on one lateral side of an axis of the stem, and
wherein the proximal part has a rim around said articular surface, said rim forming rounded corners such that there is no edge or point loading when in contact with a bone surface.

The arrangement of the proximal part, particularly the articular surface and the rim, provide for effective sliding engagement with the trapezium even if the joint is subluxed, and the implant surgery does not try to re-align the joint, rather working with the anatomy.

In some preferred examples, the proximal articular surface is configured so that a line which is normal to a chord of the articular surface across the widest orientation of the implant and a longitudinal centre line of the stem subtend an angle A in the range of 15°to 60°.

In some preferred examples, the proximal concave articular surface has a radius of curvature (R) in the range of 10 mm to 30 mm.

In some preferred examples, the apparatus further comprising a rasp tool having an internal rasp surface curvature radius matching the concave proximal articular surface.

In some preferred examples, the rasp tool internal surface has a radius in the range of 10 mm to 25 mm, optionally approximately a 12.5 mm.

In some preferred examples, the rasp tool has a dual function comprising one side providing a broach function and another side providing said rasp function.

In some preferred examples, the intramedullary part and the proximal part are modules which are joined together.

In some preferred examples, the intramedullary part comprises a body and a coating.

In some preferred examples, the intramedullary part comprises any one or more material selected from CoCr, Ti, PEEK, CF-PEEK, and Pyro Carbon.

In some preferred examples, the coating comprises a titanium plasma material coating, to provide an interference fit and promote osseointegration.

In some preferred examples, the coating material comprises commercially pure titanium (CPTi). In some preferred examples, the intramedullary part body has a surface roughness beneath the coating which has an Ra value greater than 6.0 μm.

In some preferred examples, a surface of the coating, which engages the bone has a surface roughness in the range of 20 μm to 40 μm.

In some preferred examples, the coating has a distribution with an elongate pattern and a continuous band around a proximal base area of the intramedullary body, to combat hoop stress.

In some preferred examples, there are a plurality of proximal parts each of a different proximal surface shape and at least one intramedullary part, wherein a selected proximal part may be engaged with the intramedullary part to provide an implant.

In some preferred examples, at least one proximal part and a plurality of intramedullary parts each of which is configured to suit a different bone size and/or shape, wherein the proximal part may be engaged with a selected intramedullary part to provide an implant.

In some preferred examples, the apparatus comprising a plurality of proximal parts each of a different proximal surface shape and a plurality of intramedullary parts each of which is configured to suit a different bone size and/or shape, wherein a selected proximal part may be engaged with a selected intramedullary part to provide an implant.

In some preferred examples, the parts are configured to be joined by an interference fit such as a Morse taper or a screw.

We also describe a method of providing a hemi-arthroplasty implant in a subluxed CMC joint, the method comprising performing a metacarpal resection, performing a trapezium re-shaping, and inserting the intramedullary body of an apparatus of any example into the metacarpal canal without realigning the joint.

In some preferred examples, said re-shaping is performed with a rasping tool.

In some preferred examples, the resection removes a length of the proximal end of the metacarpal in the range of 3 mm to 8 mm, preferably 3 mm to 5 mm.

In some preferred examples, the trapezium is re-shaped to have a curvature matching the curvature of said proximal part articular surface.

In some preferred examples, the method is performed with an apparatus having a plurality of modular proximal parts of different configurations, wherein an implant proximal part is chosen according to the degree of sub-luxation of the CMC joint.

We also describe an implant apparatus comprising an implant comprising an intramedullary part and a proximal part, said parts being fixed together or integral, said proximal part having a proximal articular surface which is curved with a concave portion for direct contact and sliding engagement with a distal surface of a proximal bone.

In one example, the articular surface is curved in an asymmetric manner with a part of the proximal surface extending more proximally on one lateral side of an axis of the stem.

In one example, the proximal part has rounded corners such that there is no edge or point loading when in contact with a bone surface. In one example, the proximal part articular surface includes a concave portion for engagement with a bone distal convex portion.

In one example, the intramedullary part is configured for intramedullary engagement in a human metacarpal. In one example, the apparatus further comprises a rasp tool having an internal rasp surface curvature radius matching an angle matching the proximal articular surface.

In one example, the intramedullary part and the proximal part are modules which are joined to provide the stem. In one example, the apparatus comprises a plurality of proximal part modules each of a different proximal surface shape. In one example, the modules are configured to be joined by an interference fit such as a Morse taper or a screw.

In one example, the intramedullary part comprises a body and a coating. In one example, the rasp tool has a radius in the range of 10 mm to 25 mm, optionally approximately a 12.5 mm.

In one example, the rasp tool has a dual function comprising one side providing a broach function and another side providing a rasp function.

In one example, the intramedullary part comprises any one or more material selected from CoCr, Ti, PEEK, and Pyro Carbon.

In one example, the intramedullary body comprises CF-PEEK material.

In one example, the coating comprises a titanium plasma material coating, to provide an interference fit and promote osseointegration.

In one example, wherein the coating material comprises commercially pure titanium (CPTi).

In one example, the intramedullary part body has a surface roughness beneath the coating which has an Ra value greater than 6.0 μm.

In one example, a surface of the coating has a surface roughness in the range of 20 μm to 40 μm.

In one example, the coating has a distribution with an elongate pattern and a continuous band around a proximal base area of the intramedullary body, to combat hoop stress.

Preferably, the proximal articular surface is configured so that a line which is normal to a chord of the articular surface across the widest orientation of the implant and a longitudinal centre line of the stem subtend an angle in the range of 15° to 60°.

In one example, the proximal articular surface has a radius of curvature in the range of 10 mm to 30 mm.

We also describe a kit comprising a plurality of proximal parts each of a different proximal surface shape and at least one intramedullary part, wherein a selected proximal part may be engaged with the intramedullary part to provide an implant of any example.

We also describe a kit comprising at least one proximal part and a plurality of intramedullary parts each of which is configured to suit a different bone size and/or shape, wherein the proximal part may be engaged with a selected intramedullary part to provide an implant of any example.

We also describe a kit comprising a plurality of proximal parts each of a different proximal surface shape and a plurality of intramedullary parts each of which is configured to suit a different bone size and/or shape, wherein a selected proximal part may be engaged with a selected intramedullary part to provide an implant of any example.

For any of the above kits, the parts may be configured to be joined to by an interference fit such as a Morse taper or a screw.

We also describe a method of providing a hemi-arthroplasty implant in a CMC joint, the method comprising performing a metacarpal resection and a trapezium re-shaping and inserting the intramedullary body of an apparatus of any example into the metacarpal canal.

In one example, said re-shaping is performed with a rasping tool.

### DETAILED DESCRIPTION OF THE INVENTION

The invention will be more clearly understood from the following description of some embodiments thereof, given by way of example only with reference to the accompanying drawings in which:

FIGS. 1 to 3 are images of prior art bone joint situations as set out above in the introductory passages,

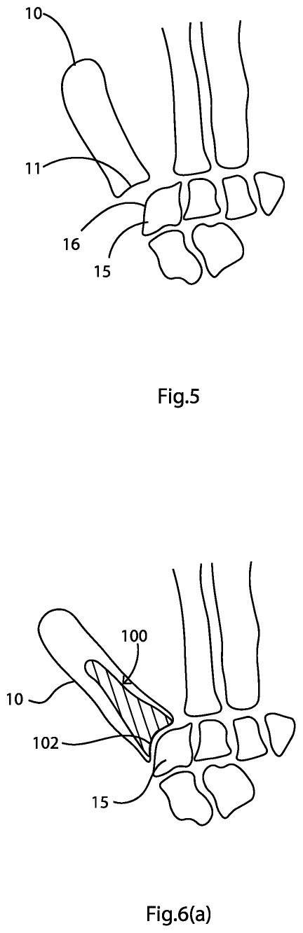

FIG. 4 shows a metacarpal resection and a trapezium re-shaping using a rasping tool in preparation for insertion of an implant of the invention,

FIG. 5 is a diagram showing the metacarpal and the trapezium after resection and re-modelling respectively,

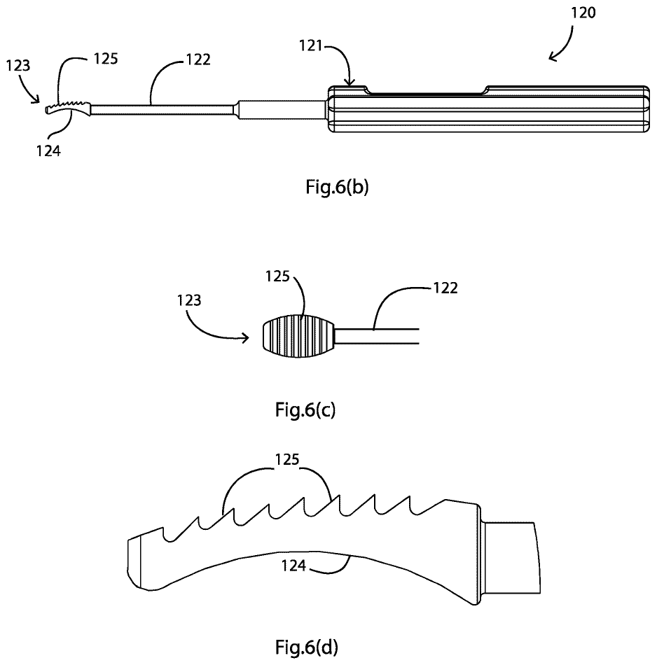

FIG. 6(a) is a diagram showing the implant medullary stem in place after surgery, with the stem proximal end matching the distal surface of the trapezium after it has been re-modelled using the rasp tool, and FIG. 6(b), (c), and (d) are respectively a side view of a rasp tool, a front view of broach teeth of the head of the rasp tool, and an enlarged view showing both a rasp surface and broach teeth,

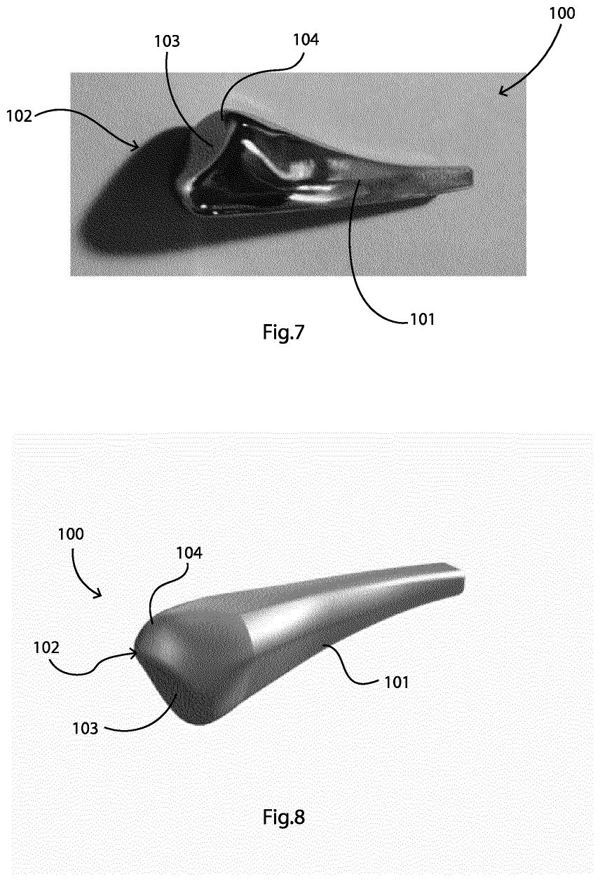

FIG. 7 is a photographic image of the implant, and FIG. 8 is a 3D model representation of the implant,

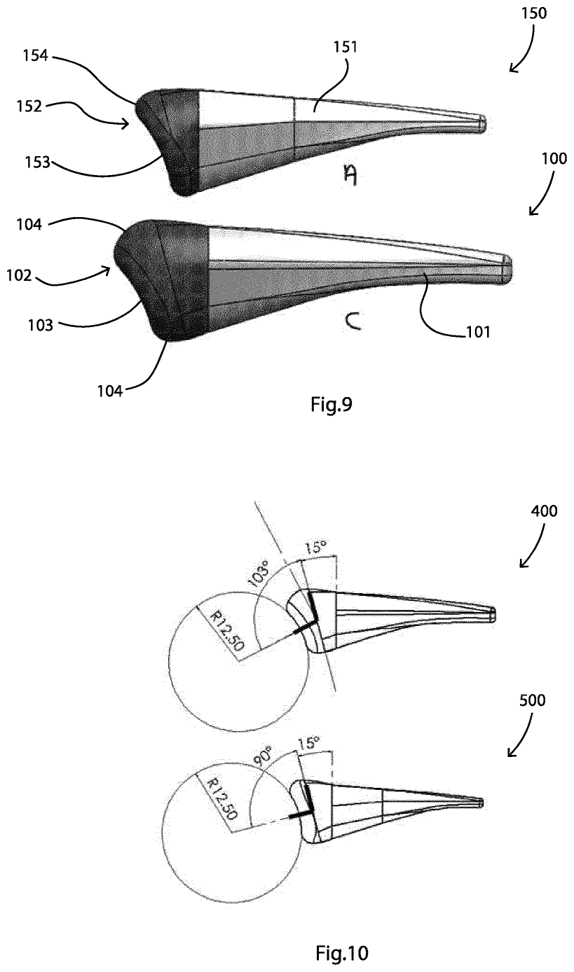

FIG. 9 is a pair of side views of alternative implants, with different configurations, top diagram being Size A and bottom diagram being Size C;

FIG. 10 is a pair of side views showing further alternative implants and key dimensional parameters of them, showing the alteration in angulation i.e. the offset, possible by alteration of the degree of alignment of the proximal portion to the distal portion;

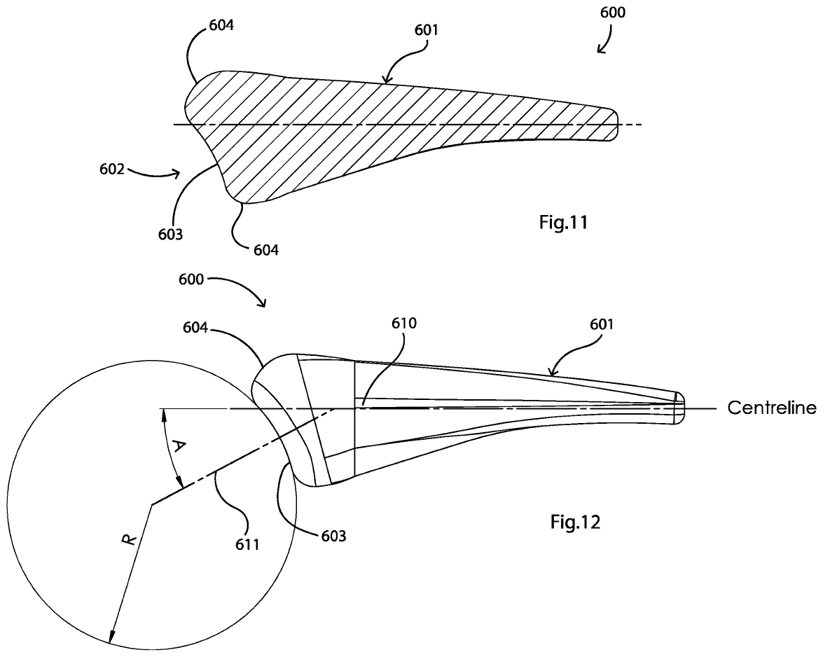

FIG. 11 is a longitudinal sectional view of an implant, and FIG. 12 is the same view with hatching removed for clarity and showing parameters for the configuration of the implant; and

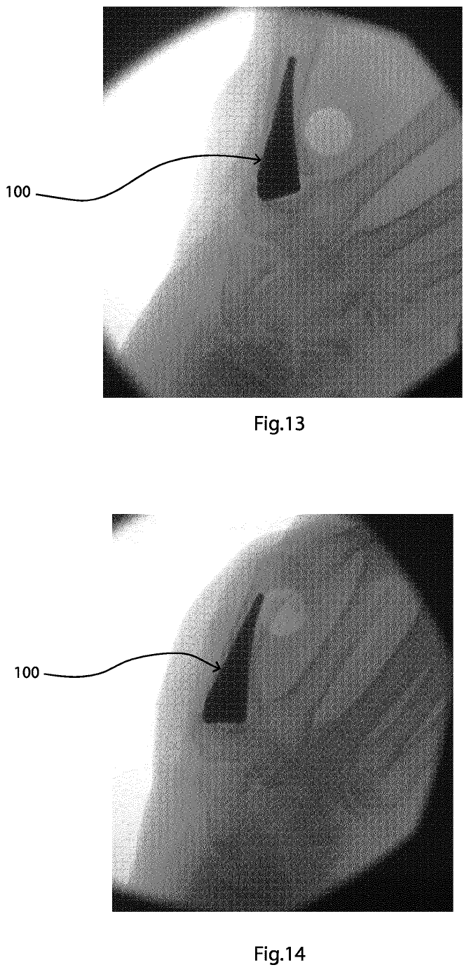

FIGS. 13 and 14 are X-ray images of an implant of the invention when implanted, the images showing different thumb positions.

### DETAILED DESCRIPTION OF THE INVENTION

The invention addresses the problem that prior implants for the CMC joint tend to be designed to function when the joint is normally aligned. Unfortunately, a degenerated CMC joint tends not to be aligned, or even after re-alignment, and tends to fall back into its old non-aligned position. This causes failure of implants as the joint takes any implant with it as it moves out of alignment.

We describe here a “monoblock” implant to function in the non-aligned joint. The invention takes into account the fundamental biomechanics of the thumb base joint, and the most common failure modes of existing implants into account for the treatment of CMC joint arthritis. The implant is most advantageous for a CMC joint that has some laxity and dorsal subluxation. The implant does not restore the pre-disease biomechanics of the joint and the normal alignment of the bones, but instead provides a treatment for the joint in its subluxed state. By doing so, the invention aims to remove the failure modes of many existing implants. The implant has specific features to ensure that the implant functions in a subluxed joint post implantation.

Referring to FIG. 4 interrupted line 1, a depth varying in the range of approximately 3 mm to 8 mm is removed from the proximal base of the metacarpal bone, termed a “resection”. In many cases a depth in the range of 3 mm to 5 mm is preferred. In some examples, a rasp tool (such as described in our prior published specification WO2020/229143) is then used to shape the trapezium to a general saddle shape on the dorsal aspect of the bone, as shown by the interrupted line 2 of FIG. 4. The surgeon may refer to pre-operative X rays to determine where the metacarpal is likely to revert to post-operatively. For example, in FIG. 1 the X-ray indicates the degree of subluxation that should be accounted for when shaping the trapezium. The metacarpal stem size will be determined by the broach size.

The rasping of the trapezium may not be a requirement for the adequate functioning of the implant. If the trapezium is quite worn down already, no rasping may be needed but if there are any larger dorsal osteophytes (bony protrusions) the surgeon may choose to rasp these off to match the base of the implant to the surface of the trapezium.

FIG. 4 shows a metacarpal resection (1) and trapezium re-shaping using the rasp tool (2). FIG. 5 is a diagram showing the post-resected metacarpal 10 and re-modelled trapezium 15. The metacarpal 10 has a resection proximal surface 11 and the trapezium 15 has a distal re-modelled surface 16. The surgeon may choose to make an angulated excision of the metacarpal depending on the degree of subluxation.

FIG. 6(a) shows a medullary-shaped stem implant 100 of the invention in place with an intramedullary part and a proximal part having an articular surface 102 matching the geometry of the re-modelled trapezium 15. FIG. 6(b), (c), and (d) show a rasp tool 120 for use in this procedure. There is a handle 121, a stem 122, and a head 123 having a convex part with broach teeth 125 and a concave rasp surface 124.

The rasp 124 on the concave side may be used to sculpt the trapezium bone to the exact radius of the concave articular surface 103. The convex side of the tool head 123 carries broach-like teeth 125 which may be used to remove incidental osteophytes or other protuberances encountered in the course of sculpting the trapezium. The concave side 124 of the head 123 has rasp a construction for sculpting the trapezium. The radius of curvature of the rasp surface 124 preferably matches that of the concave articular surface 103.

As shown in FIGS. 7 and 8, the implant 100 of the invention comprises an intramedullary part 101 for insertion into a bone such as the metacarpal and a proximal part 102 having a proximal articular surface 103 which is curved in a generally concave manner for direct contact and sliding engagement with a generally convex distal surface of a proximal bone such as the trapezium. The proximal articular surface 103 is curved in an asymmetric manner with a part of the proximal surface extending more proximally on one lateral side of an axis of the stem. In general, it is at an angle to the longitudinal axis of the stem, as described in more detail below. The proximal part has rounded corners 104 without any sharp edges. These rounded corners are of a convex shape, with varying degrees of curvature and not necessarily circular in section. The transition from the shallow articular surface 103 to the surface of the intramedullary part 101 is provided in a continuous manner by the convex rim 104, which does not present any sharp edges or corners.

The implant apparatus may also include a rasp tool having an angle matching the articular proximal surface of the implant.

By remodelling the trapezium, the dorsal side of the bone has an angle such as 12.5° against which the implant can articulate with the joint in a subluxed position. As shown in these figures, the joint remains in the pre-operative subluxed position, and the forces on the joint are maintained at the pre-operative equilibrium. This avoids the creation of dorsal subluxation forces which may cause a malfunction of the implant.

FIG. 9 is a pair of side views of the implant 100 (Size C) and of an implant 150 (Size A), the latter having an intramedullary body 151 and a proximal part 152 with an articular proximal surface 153 with the desired proximal face shape. The outer edge of the articular surface 153 is formed by a rounded convex rim 154, viewed as convex rounded corners in any longitudinal section.

FIG. 10 shows images of implants 400 and 500 with different degrees of angulation. The implant 400 has 103° between the centre of the proximal surface and a 15° angulation line relative to a normal to the centre line. This would cause the implant to be more heavily weighted on the dorsal side and potentially sit further dorsally in the joint. This may be optional for some joint anatomy. The implant 500 has a 90° angle between the midpoint of the proximal surface and hence would sit more centrally on the trapezial surface. This may be more useful if the joint is less subluxed than may be the case for a more subluxed joint where the stem 400 may be more preferred.

It will be noted that there are rounded edges on the proximal part to ensure that there is no point loading. The rounded edge surrounds the concave part, forming a convex rim for continuity of the stem surface to the concave surface. This allows very effective sliding engagement of the implant articular surface 103 over the trapezium. Also, the intramedullary body (stem part) is shaped to match the internal anatomy of the metacarpal, the proximal part being shaped to articulate with the trapezium or the dorsal aspect of the trapezium in a subluxed joint.

In order to provide modularity and attendant alternate degrees of articulation, the implant intramedullary and proximal parts may be provided in two parts. The two elements may be joined by an interference fit such as a Morse taper, or a screw or other locking mechanism. This allows choice of an optimum stem and separately choice of an optimum proximal part to suit the patient's anatomy.

Referring to FIG. 11 an implant 600 has a stem 601 and a proximal part 602, which in this case are integral. The proximal part has an articular concave surface 603 surrounded by a rounded convex edge or rim 604. In all embodiments the rounded rim provides smooth continuity from the articular surface to the surface of the stem. As viewed in FIG. 12 the articular surface has an average radius of curvature R in the range of 10 mm to 30 mm.

The centreline of the stem is indicated as 610. A line 611 is also shown, which is normal to a chord of the articular surface 603 across the widest orientation of the implant. The angle between the lines 610 and 611 is denoted “A” and is preferably in the range of 15° to 60°. The greater this angle the greater the extent of the offset.

The greater the degree of angulation of the articular surface, the more appropriate it is for use in a more subluxed joint i.e. an angulation of 15 degrees is minimal and may be used in a minimally subluxed joint, while a 60 degree angle will be used in a very subluxed joint. In addition, as the implant may be modular, the surgeon can choose to use one stem size, and one head size based on the size of the metacarpal and the degree of subluxation, tailoring the implant to the biomechanics of the subluxed joint. Alternatively, the surgeon can rasp the trapezium to 12.5 degrees and use that head size if preferred. In summary the surgeon can tailor the implant modularity to the joint, or tailor the joint to the implant.

FIGS. 13 and 14 are X-ray images of the implant 100 when implanted, the images showing different thumb positions.

The implant is designed to function when the joint is subluxed, unlike previous implants which are designed to function when the joint has been re-aligned to a normal anatomical position. The implant is configured to minimise the impact of the positioning of the implant. If the joint is subluxed or becomes subluxed, the implant functions.

The implant is curved and shaped such that each surface that potentially articulates with bone is curved for an optimal distribution of joint forces. The medullary shape of the implant is approximated to the known approximate internal geometry of the metacarpal medullary canal. This provides for an improved interference fit, reducing the risk of loosening or pistoning of the stem. The stem is provided in a number, preferably five, sizes to provide size options based on the variance of metacarpal bones sizes.

The intramedullary body preferably has a TPS (Titanium Plasma Spray) coating along the dorsal aspect and in a ring at the proximal base to improve the interference fit of the stem.

In one example, the rasp tool has a 12.5 mm radius, and in general it is preferred that the radius is in the range of 10 mm to 25 mm. The rasp tool has a dual function with one side providing a broach function while the other provides a rasp function. This facilitates optimal re-modelling of the trapezial surface, which may include both hard outer cortical bone, and softer cancellous bone.

For example, if it is suitable to reshape the trapezium in many cases, the surgeon may use a range of rasp tools of varying radii to optimally sculpt the trapezium surface. A toolset including any or all of the tools described in WO2020/229143 may be used.

Preferably, the proximal end articular surface matches the geometry of the rasp exactly, but this is not essential. This provides for an optimal congruence between the modified trapezium bone surface and the base of the implant stem. The invention may comprise a kit of an implant and a rasp tool with a matching angle for optimal re-modelling of the trapezium to provide a distal surface matching the proximal surface of the stem.

One or both parts of the implant parts may comprise a body of from CoCr, Ti, PEEK, Pyro Carbon or any such material with good wear properties in relation to bone. The implant may comprise different materials to optimise stability in the metacarpal and wear on the trapezium.

Advantageously, the implant is configured to function in a sublux or non-aligned joint. The implant is advantageously configured to be placed, and function, in a subluxed non-aligned joint. This is a major improvement over the prior approach of implants being provided to realign the joint. The shallow concave articular surface allows accommodation of the current patient anatomy, whereas the prior art approach is to surround the trapezium in a manner which re-aligns the joint.

As described, the invention is particularly advantageous for the CMC joint.

The proximal geometry is such that if the joint is in a subluxed position, the implant articulations against the trapezium should still provide a smooth articular surface allowing the implant to function and the patient to experience pain reduction.

The intramedullary part of the implant which sits within the metacarpal and does not articulate with surrounding bones may have a surface coating applied to facilitate an interference-fit into the medullary canal of the metacarpal, and to facilitate bony ingrowth when the stem portion is in place. An example of such a coating would be Titanium Plasma Spray (TPS). The surface of the coating, which engages the bone may have a surface roughness of 30 μm, and more generally it is preferred that it be in the range of 20 μm to 40 μm. This is particularly effective for osteointegration. Such a coating is preferably not applied over all of the bone-contacting surface of the integral body. The preferred coating distribution is along a line extending distally on the intramedullary part, such as along a dorsal segment. In this example the intramedullary part has a TPS coating as described above extending distally in an elongated pattern from the proximal end to the distal end, although in other examples this elongate pattern does not extend the full length of the stem. It is also preferable that there be a continuous band around the proximal base area of the intramedullary part to combat hoop stress.

Additionally, the non-articular surface of the implant may be grit blasted to provide a localised surface roughness to enhance an interference fit between the metacarpal medullary canal and the stem.

One or both parts of the implant may be fabricated by CNC machining, additive manufacturing processes, or functional gradient manufacturing. Where the implant is a one-piece configuration, it would be suited to additive manufacturing techniques which may include a surface geometry to provide for an improved interference fit.

Also, as the implant may be fabricated by functional gradient manufacturing the proximal part may be of a different composition in terms of density, materials, or material properties than the distal end of the implant. This may provide a more physiologically correct matching of the implant properties and the bone properties. For example, this better matching of material properties may avoid bony stresses such as Wolff forces which can lead to implant movement post-placement and ultimately implant failure.

It will be appreciated that the implant is always a unitary element, even though the intramedullary and proximal parts may be manufactured separately and then joined together to provide the implant which is used. Indeed, the parts may be selected by the surgeon from a kit with a set of proximal parts with different articular surfaces and/or a set of candidate intramedullary parts to suit different bone sizes, and the selected parts jointed by the surgeon to provide the unitary implant. For any of the kits, the parts may be configured to be joined to by an interference fit such as a Morse taper or a screw.

Also, it will be appreciated that the invention allows surgery for painful joints in an approach in which the surgeon is not trying to counter the patient's anatomy, but rather working with it. Hence for example an older patient may have the benefit of relief from the pain of arthritis and improved hand function, but without the potential discomfort involved in attempting to realign the joint.

Components of embodiments can be employed in other embodiments in a manner as would be understood by a person of ordinary skill in the art. The invention is not limited to the embodiments described but may be varied in construction and detail.

1. An implant apparatus comprising a stem implant, the stem implant comprising:
an intramedullary part configured for intramedullary engagement in a human metacarpal, and
a proximal part,
wherein said parts are fixed together or integral, said proximal part has a proximal articular surface which is curved with a concave portion for direct contact and sliding engagement with a distal surface of a trapezium,
wherein said articular surface is curved in an asymmetric manner with a part of the articular surface extending more proximally on one lateral side of an axis of the stem, and
wherein the proximal part has a rim around said articular surface, said rim forming rounded corners such that there is no edge or point loading when in contact with a bone surface.

2. The implant apparatus as claimed in claim 1, wherein the proximal articular surface is configured so that a line which is normal to a chord of the articular surface across the widest orientation of the implant and a longitudinal centre line of the stem subtend an angle A in a range of 15° to 60°.

3. The implant apparatus as claimed in claim 1, wherein the proximal concave articular surface has a radius of curvature in a range of 10 mm to 30 mm.

4. The implant apparatus as claimed in claim 1, further comprising a rasp tool having an internal rasp surface curvature radius matching the concave proximal articular surface.

5. The implant apparatus as claimed in claim 4, wherein the rasp tool internal surface has a radius in a range of 10 mm to 25 mm.

6. The implant apparatus as claimed in claim 4, wherein the rasp tool has a dual function comprising one side providing a broach function and another side providing said rasp function.

7. The implant apparatus as claimed in preceding claim 1, wherein the intramedullary part and the proximal part are modules which are joined together.

8. The implant apparatus as claimed in claim 1, wherein the intramedullary part comprises a body and a coating.

9. The implant apparatus as claimed in claim 1, wherein the intramedullary part comprises one or more materials selected from CoCr, Ti, PEEK, CF-PEEK, and Pyro Carbon.

10. The implant apparatus as claimed in claim 8, wherein the coating comprises a titanium plasma material coating, to provide an interference fit and promote osseointegration.

11. (canceled)

12. The implant apparatus as claimed in claim 8, wherein the intramedullary part body has a surface roughness beneath the coating which has an Ra value greater than 6.0 μm.

13. The implant apparatus as claimed in claim 8, wherein a surface of the coating which engages the bone has a surface roughness in a range of 20 μm to 40 μm.

14. The implant apparatus as claimed in claim 8, wherein the coating has a distribution with an elongate pattern and a continuous band around a proximal base area of the intramedullary body, to combat hoop stress.

15. The implant apparatus as claimed in claim 7, further comprising a plurality of proximal parts, wherein each proximal part has a different proximal surface shape, and at least one intramedullary part wherein at least one proximal part of the plurality of proximal parts may be engaged with the at least one intramedullary part to provide an implant.

16. The implant apparatus as claimed in claim 7, comprising wherein the intramedullary part and the proximal part are modules which are joined together, and wherein the apparatus comprises at least one proximal part and a plurality of intramedullary parts, wherein each intramedullary part is configured to suit a different bone size and/or shape, and wherein the proximal part may be engaged with at least one intramedullary part from the plurality of intramedullary parts to provide an implant.

17. (canceled)

18. The implant apparatus as claimed in claim 7, wherein the intramedullary part and the proximal part are configured to be joined by an interference fit such as a Morse taper or a screw.

19. A method of providing a hemi-arthroplasty implant in a subluxed CMC joint, the method comprising:
performing a metacarpal resection,
performing a trapezium re-shaping, and
inserting an intramedullary body of an implant stem into a metacarpal canal without realigning the joint, the implant stem comprising:
an intramedullary part configured for intramedullary engagement in a human metacarpal, and
a proximal part,
wherein said parts are fixed together or integral, said proximal part has a proximal articular surface which is curved with a concave portion for direct contact and sliding engagement with a distal surface of a trapezium,
wherein said articular surface is curved in an asymmetric manner with a part of the articular surface extending more proximally on one lateral side of an axis of the stem, and
wherein the proximal part has a rim around said articular surface, said rim forming rounded corners such that there is no edge or point loading when in contact with a bone surface.

20. The method as claimed in claim 19, wherein the re-shaping step is performed with a rasping tool.

21. The method as claimed in claim 19, wherein the resection step includes removing a length of the a proximal end of the metacarpal in a range of 3 mm to 8 mm.

22. The method as claimed in claim 19, wherein the trapezium is re-shaped to have a curvature matching the curvature of said proximal part articular surface.

23. (canceled)
